(19) United States
(12) Patent Application Publication
(10) Pub. No.: US 2018/0338670 A1
(43) Pub. Date: Nov. 29, 2018

(54) LIGHT SOURCE CONTROL DEVICE, LIGHT SOURCE CONTROL METHOD, PROGRAM, AND SURGICAL SYSTEM

(71) Applicant: SONY CORPORATION, TOKYO (JP)

(72) Inventors: KENTA YAMAGUCHI, KANAGAWA (JP); HISAKAZU SHIRAKI, KANAGAWA (JP); HIROSHI ICHIKI, KANAGAWA (JP); YUKI SUGIE, KANAGAWA (JP)

(21) Appl. No.: 15/761,553

(22) PCT Filed: Sep. 23, 2016
(86) PCT No.: PCT/JP2016/077939

§ 371 Date: Mar. 20, 2018

(30) Foreign Application Priority Data
Oct. 7, 2015 (JP) 2015-199670

(51) Int. Cl. A61B 1/00; G06T 7/514; G06T 7/593; A61B 1/06

(52) U.S. Cl. CPC A61B 1/00006; G06T 7/514; G06T 7/593; A61B 1/00193; G06T 2207/30004; A61B 1/0676; G06T 2207/10028; G06T 2207/10068; G06T 2207/10012; A61B 1/0607

(57) ABSTRACT

The present disclosure relates to a light source control device, a light source control method, a program, and a surgical system that enable observation with a more appropriate exposure state. The light source control device includes a reflection direction estimation unit which estimates a reflection direction of irradiation light reflected on a surface of a subject and a control unit which controls a direction of a light source for emitting the irradiation light on the basis of the estimated reflection direction of the irradiation light. Then, on the basis of the reflection direction, the control unit performs control so that illuminance of the subject viewed from a direction in which the subject is observed becomes uniform or so that specular reflection to the direction in which the subject is observed is suppressed. The present technology is applied to, for example, a surgical system used for endoscopic surgery.

### TECHNICAL FIELD

The present disclosure relates to a light source control device, a light source control method, a program, and a surgical system, and more specifically, to a light source control device, a light source control method, a program, and a surgical system that enable observation with a more appropriate exposure state.

### BACKGROUND ART

In recent years, surgery using an endoscope has been performed instead of a traditional laparotomy in a medical field. Furthermore, it has been known that it is difficult to set an appropriate exposure state in observation with the endoscope, and a technology to uniformly irradiate a subject has been developed.

For example, Patent Document 1 discloses an endoscope apparatus which changes an irradiation light quantity and a light distribution pattern according to distance information and adjusts uneven light distribution by gain correction. In addition, Patent Document 2 discloses an endoscope apparatus which reduces the uneven light distribution (shading) by a plurality of light sources.

### CITATION LIST

### Patent Document

Patent Document 1: Japanese Patent Application Laid-Open No. 2015-8785

Patent Document 2: Japanese Patent Application Laid-Open No. 2012-245349

### SUMMARY OF THE INVENTION

### Problems to be Solved by the Invention

However, even if appropriate exposure can be realized by the technologies disclosed in Patent Documents 1 and 2 described above, reflection components are not considered in these technologies. Therefore, it has been difficult to obtain an image suitable for observation due to specular reflection.

The present disclosure has been made in view of such a situation, and enables observation with a more appropriate exposure state.

### Solutions to Problems

A light source control device according to one aspect of the present disclosure includes a reflection direction estimation unit which estimates a reflection direction in which irradiation light is reflected on a surface of a subject in a living body and a control unit which relatively controls a direction of a light source for emitting the irradiation light relative to a direction in which the subject is observed on the basis of the reflection direction of the irradiation light estimated by the reflection direction estimation unit and the direction in which the subject is observed.

A light source control method or a program according to one aspect of the present disclosure includes estimating a reflection direction in which irradiation light is reflected on a surface of a subject in a living body and relatively controlling a direction of a light source for emitting the irradiation light relative to a direction in which the subject is observed on the basis of the reflection direction of the estimated irradiation light and the direction in which the subject is observed.

A surgical system according to one aspect of the present disclosure includes a light source which irradiates an operated portion in a living body with irradiation light from a predetermined direction, an imaging unit which images the operated portion in the living body, a reflection direction estimation unit which estimates a reflection direction in which irradiation light is reflected on a surface of the operated portion, and a control unit which relatively controls a direction of the light source relative to an optical axial direction of the imaging unit on the basis of the reflection direction of the irradiation light estimated by the reflection direction estimation unit and the optical axial direction of the imaging unit.

In one aspect of the present disclosure, a reflection direction of irradiation light reflected on a surface of a subject in a living body is estimated, and a direction of a light source for emitting the irradiation light is relatively controlled relative to a direction in which the subject is observed on the basis of the reflection direction and the direction in which the subject is observed.

### Effects of the Invention

According to one aspect of the present disclosure, observation with a more appropriate exposure state can be realized.

### BRIEF DESCRIPTION OF DRAWINGS

FIG. 1 is a diagram of an exemplary overall configuration of an embodiment of an endoscopic surgical system to which the present technology is applied.

FIG. 2 is an explanatory diagram of an exemplary hardware configuration of a CCU.

FIG. 3 is a diagram to describe an exposure state in a natural environment.

FIG. 4 is a diagram to describe an exposure state of an endoscope.

FIG. 5 is a diagram to describe a structure of a traditional endoscope.

FIG. 6 is a diagram of the Phong reflection model.

FIG. 7 is a diagram of an appearance of a rigid endoscope including a plurality of light sources.

FIG. 8 is a block diagram of an exemplary configuration of a light source control system.

FIG. 9 is a diagram to describe a method of obtaining distance information by using stereoscopic image data and stereo matching.

FIG. 10 is a diagram to describe a method of obtaining the distance information by using the stereoscopic image data and the stereo matching.

FIG. 11 is a diagram to describe processing of estimating an inclination of a subject.

FIG. 12 is a diagram to describe a method of estimating a light source direction so that the subject is uniformly illuminated.

FIG. 13 is a diagram to describe a method of estimating the light source direction so as to minimize specular reflection.

FIG. 14 is a flowchart to describe light source control processing.

FIG. 15 is a flowchart to describe the processing of estimating the inclination of the subject.

FIG. 16 is a flowchart to describe processing of estimating an appropriate light source direction.

FIG. 17 is a diagram of a normal distribution.

FIG. 18 is a block diagram of an exemplary configuration of an embodiment of a computer to which the present technology is applied.

### MODE FOR CARRYING OUT THE INVENTION

A specific embodiment to which the present technology has been applied will be described below in detail with reference to the drawings.

<Exemplary Configuration of Endoscopic Surgical System>

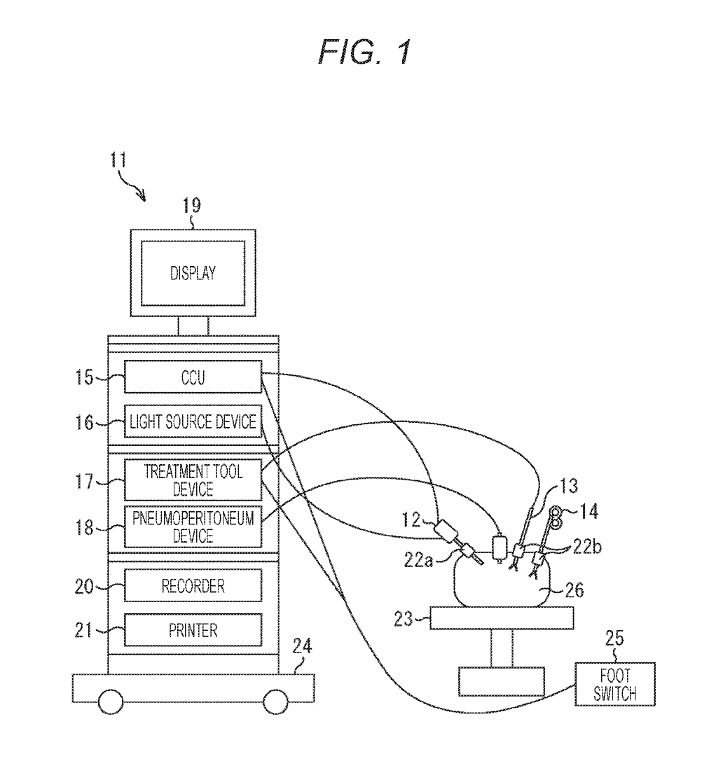

FIG. 1 is a diagram of an exemplary overall configuration of an endoscopic surgical system as a surgical system according to the present technology.

In recent years, endoscopic surgery has been performed instead of a traditional laparotomy in a medical field. For example, in a case of abdominal surgery, an endoscopic surgical system 11 which is arranged in an operation room as illustrated in FIG. 1 is used. Instead of opening an abdominal wall to perform laparotomy as in a traditional method, opening devices referred to as trocars 22a and 22b are attached at some positions in the abdominal wall, and a camera head portion 12 (simply referred to as camera head below) of a laparoscope (referred to as endoscope), an energy treatment tool 13, a forceps 14, and the like are inserted into a body from holes provided in the trocars 22a and 22b. Then, while looking at an image of an affected part (tumor or the like) 26 video-imaged by the camera head portion 12 in real time, treatment such as cutting away the affected part 26 is performed with the energy treatment tool 13 and the like. The camera head portion 12, the energy treatment tool 13, and the forceps 14 are held by an operator, an assistant, a scopist, a robot or the like.

In the operation room where such endoscopic surgery is performed, a cart 24 on which devices for the endoscopic surgery are mounted, a bed for patient 23 on which a patient lies, a foot switch 25, and the like are arranged. On the cart 24, for example, devices such as a camera control unit (CCU) 15, a light source device 16, a treatment tool device 17, a pneumoperitoneum device 18, a display 19, a recorder 20, and a printer 21 are mounted as medical devices.

An image signal of the affected part 26 imaged through an observation optical system of the camera head portion 12 is transmitted to the CCU 15 via a camera cable, and the signal is processed in the CCU 15. After that, the signal is output to the display 19, and an endoscopic image of the affected part 26 is displayed. The CCU 15 may be connected to the camera head portion 12 via the camera cable, or may be wirelessly connected to the camera head portion 12.

The light source device 16 is connected to the camera head portion 12 via a light guide cable, can irradiate the affected part 26 by switching light beams having various wavelengths. The treatment tool device 17 is, for example, a high-frequency output device which outputs a high-frequency current to the energy treatment tool 13 which cuts the affected part 26 by using electric heat.

The pneumoperitoneum device 18 includes an air supply and suction unit and supplies air to, for example, an abdominal region in the patient's body. The foot switch 25 controls the CCU 15, the treatment tool device 17, and the like with a foot operation by the operator, the assistant, and the like as a trigger signal.

In the endoscopic surgical system 11 configured in this way, an operation by the energy treatment tool 13 and the forceps 14 is imaged by the camera head portion 12, and signal processing is performed to the image signal in the CCU 15. The affected part 26 can be observed in the image to which the signal processing has been performed.

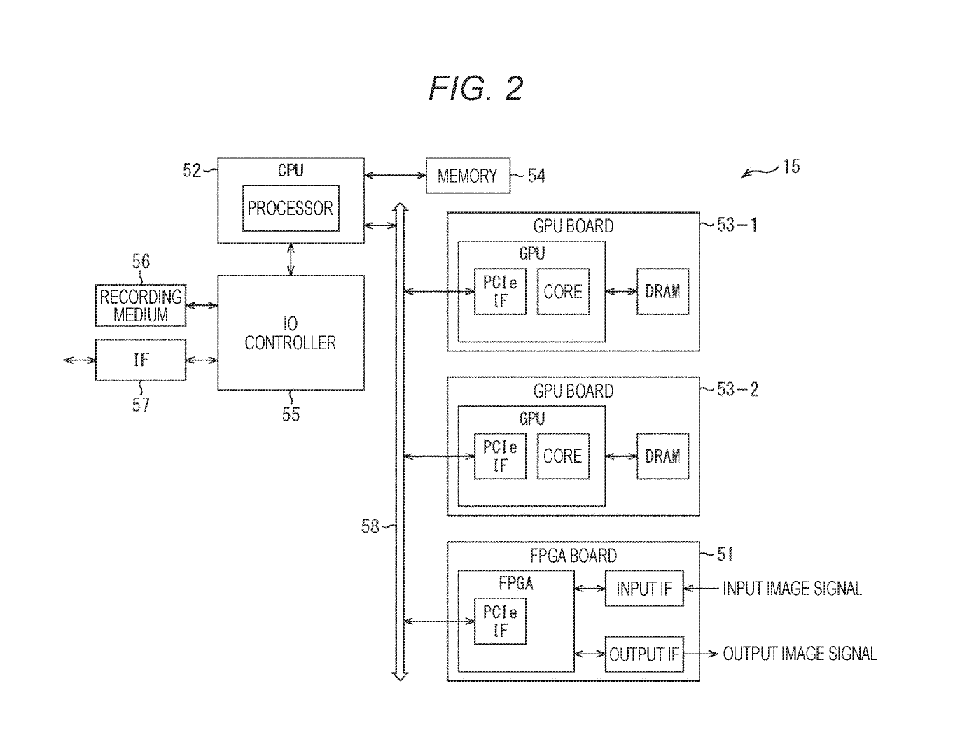

FIG. 2 is an explanatory diagram of an exemplary hardware configuration of the CCU 15 in FIG. 1. The CCU 15 includes, for example, a FPGA board 51, a CPU 52, GPU boards 53-1 and 53-2, a memory 54, an IO controller 55, a recording medium 56, and an interface 57. Furthermore, the FPGA board 51, the CPU 52, and the GPU boards 53-1 and 53-2 are connected to each other, for example, by a bus 58. The FPGA board 51 has, for example, a field-programmable gate array (FPGA), an input interface to which an input image signal is input from the camera head portion 12 in FIG. 1, and an output interface which outputs an output image signal to the display 9 in FIG. 1.

The CPU 52 and the GPU boards 53-1 and 53-2 perform various processing by executing various software such as related software, for example. The CPU 52 includes a processor. Each of the GPU boards 53-1 and 53-2 includes a graphics processing unit (GPU) and a dynamic random access memory (DRAM).

The memory 54 stores various data such as data corresponding to the input image signal from the camera head portion 12 and data corresponding to the output image signal to the display 19, for example. The CPU 52 serves to control reading and writing various data from and to the memory 54.

The central processing unit (CPU) 52 divides the image data stored in the memory 54 according to the data stored in the memory 54 and processing capabilities and processing contents of the GPU boards 53-1 and 53-2. Then, each GPU of the GPU boards 53-1 and 53-2 performs predetermined processing to the data which is divided and supplied and outputs the processing result to the CPU 52.

For example, the input output (IO) controller 55 serves to control transmission of signals between the CPU 52 and the recording medium 56, and the interface 57.

The recording medium 56 functions as a storage unit (not shown), and stores various data such as image data and various applications. Here, as the recording medium 56, for example, a solid state drive (SSD) or the like is exemplified. Furthermore, the recording medium 56 may be detachable from the CCU 15.

As the interface 57, for example, a universal serial bus (USB) terminal, a processing circuit, a local area network (LAN) terminal, a transmission/reception circuit, and the like are exemplified.

Note that the hardware configuration of the CCU 15 is not limited to the configuration illustrated in FIG. 2. For example, an example having the two GPU boards 53-1 and 53-2 is illustrated in FIG. 2. However, the number of GPU boards may be equal to or more than two. Furthermore, in a case where the CPU 52 has the function of the GPU, the CCU 15 does not have to include the GPU boards 53-1 and 53-2.

By the way, by using the endoscopic surgical system 11, a surgical technique that suppresses invasiveness, which is a major disadvantage in surgical operations, can be realized. Whereas, a universal problem in endoscopic observation is difficulty in setting the exposure.

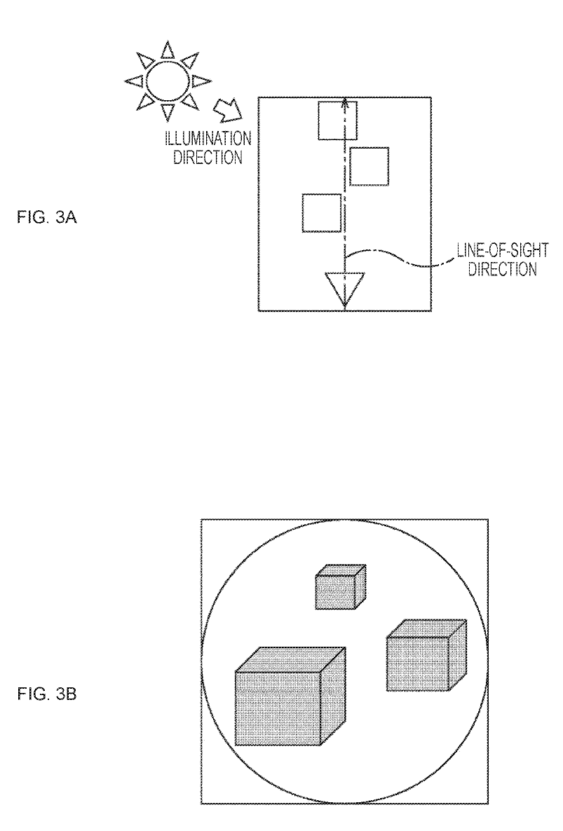

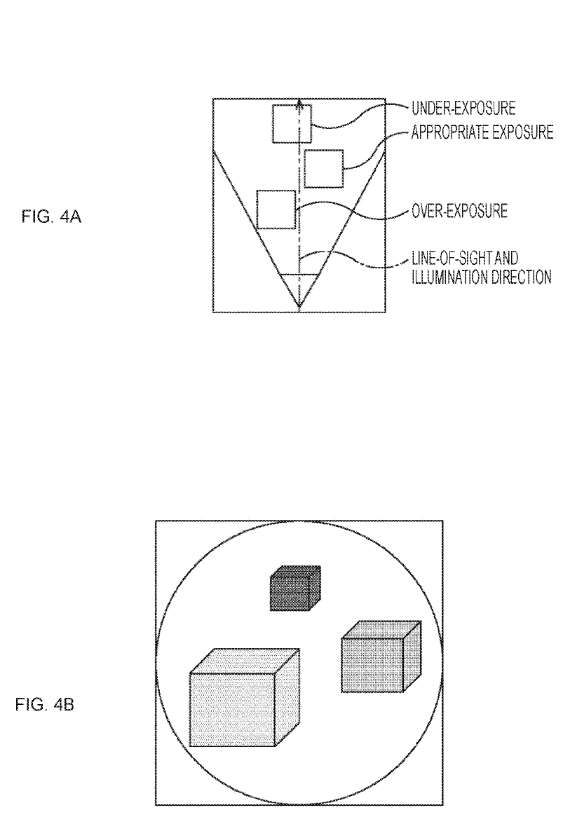

With reference to FIGS. 3 and 4, a difference between the exposure state in the natural environment and the exposure state of the endoscope will be described.

In A of FIG. 3, a relationship between a line-of-sight direction and an illumination direction in the natural environment is illustrated, and in B of FIG. 3, a state of brightness viewed from the line-of-sight direction in A of FIG. 3 is illustrated. Similarly, in A of FIG. 4, a relationship between a line-of-sight direction and an illumination direction of the endoscope is illustrated, and in B of FIG. 4, a state of brightness viewed from the line-of-sight direction in A of FIG. 4 is illustrated.

For example, as illustrated in FIG. 3, in a state where the subject is illuminated with uniform illuminance by sunlight as in the natural environment, if the objects which are uniformly irradiated with the sunlight have the same reflectance, the objects having the equal brightness are seen from the line-of-sight direction regardless of the distance.

On the other hand, as illustrated in FIG. 4, in the observation by the endoscope, the subject is unevenly illuminated by the light source. Therefore, even the objects have the same reflectance, the illuminance of the subject is changed according to the distance, and it is difficult to appropriately expose the whole field of view. That is, the short distance causes over-exposure, and the long distance causes under-exposure. The subject is displayed in the eyes with different brightness according to the distance.

Therefore, the technology disclosed in Patent Documents 1 and 2 to obtain appropriate exposure has been conventionally developed. However, although the above technology can change the light quantity and the light distribution, reflection components are not considered. Therefore, there is a possibility to image an image which has an appropriate illuminance and is not suitable for observation.

That is, from the structural reasons of traditional endoscopes, specular reflection components have been easily generated in the observation by a medical endoscope.

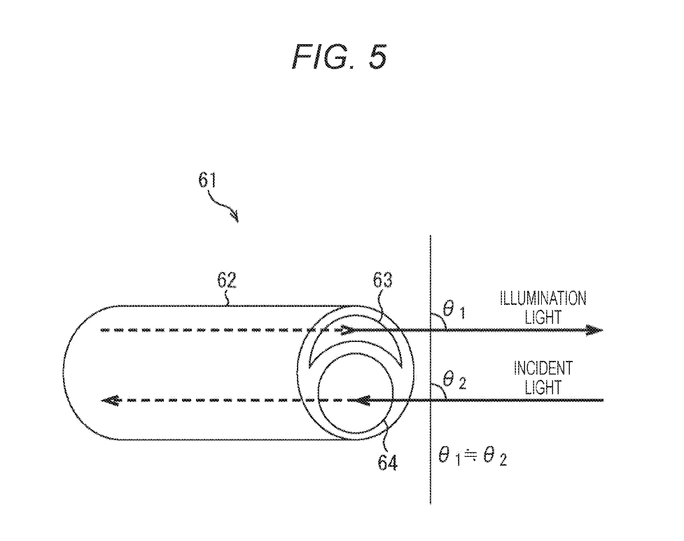

For example, as illustrated in FIG. 5, a traditional endoscope 61 has a structure in which a pair of an illumination window 63 and an observation window 64 is arranged in a front end surface of a lens barrel 62. Irradiation light from the light source is emitted from the illumination window 63 to the subject through a light guide portion. In the reflected light which is the irradiation light reflected by the subject, incident light which enters the observation window 64 is transmitted to an imaging unit.

In the endoscope 61 having such a configuration, an irradiation light vector 61 from the light source and an incident light vector θ₂ to the imaging unit are substantially parallel (θ₁≈θ₂). Therefore, if the specular reflection occurs on the surface of the subject at a high frequency, the specular reflection prevents the observation. In addition, the specular reflection causes a large stress on the eyes of the observer in some cases. Therefore, to provide an appropriate observation environment, it is necessary to consider the specular reflection components. However, as described above, the technologies disclosed in Patent Documents 1 and 2 only control the light source with an appropriate illuminance so as to suppress unevenness of the illumination (shading), and the specular reflection has not been considered. Therefore, depending on the result of the control, there has been a possibility to cause more specular reflection.

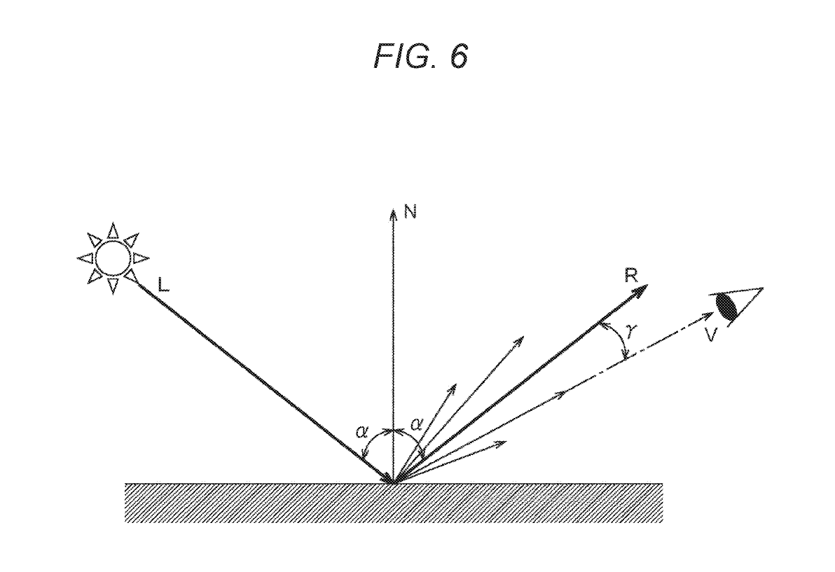

Here, with reference to FIG. 6, the Phong reflection model will be described.

For example, as illustrated in FIG. 6, at a predetermined point on a plane of a normal vector N, the light entered with an incident direction vector L which is an incident angle α is reflected with a reflection direction vector R which is an angle of reflection α, and it is assumed that a line-of-sight vector V with respect to the reflection direction vector R form an angle Υ. In this case, an intensity I_{d} of a diffuse reflection component of the reflected light is expressed by the following formula (1) by using an intensity Iᵢ of the incident light, a diffuse reflectance K_{d}, and an angle of reflection α.

[Formula 1]

I_{d}=I₁×K_{d}×cosα (1)

As indicated in Formula (1), the intensity I_{d} of the diffuse reflection component varies according to the reflection direction (angle of reflection α).

Similarly, an intensity S of specular reflection light of the reflected light is expressed by the following formula (2) by using the intensity Iᵢ of the incident light, a specular reflectance W (constant), a highlight coefficient n, and the angle Υ.

[Formula 2]

S=Iᵢ×W×cosⁿΥ (2)

As indicated in Formula (2), the intensity S of the specular reflection light decreases according to the angle Υ formed by the line-of-sight direction vector V and the reflection direction vector R.

Furthermore, in the Phong reflection model, a shade I at this point is expressed by the following formula (3) by using an intensity Iₐ and a reflectance Kₐ of environment light.

[Formula 3]

I=I_{d}+S+Iₐ=Kₐ (3)

Here, as indicated in Formula (1), since the intensity I_{d} of the diffuse reflection component changes according to the reflection direction, to calculate an appropriate illuminance, it is necessary to use not only the distance information but also the reflection direction.

Therefore, the endoscopic surgical system 11 can provide an image suitable for observation by using the distance information from the light source to the subject and the light source of which the direction can be changed.

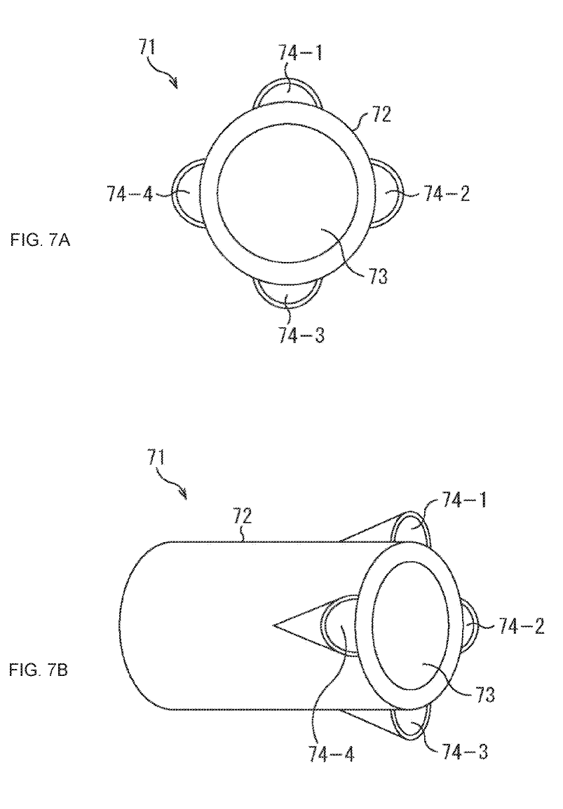

FIG. 7 is a diagram of an appearance of a rigid endoscope including the plurality of light sources used in the endoscopic surgical system 11. In A of FIG. 7, an exemplary configuration of an appearance of a rigid endoscope 71 viewed from the front direction is illustrated, and in B of FIG. 7, an exemplary configuration of the appearance of the rigid endoscope 71 viewed from the front oblique direction is illustrated.

As illustrated in FIG. 7, the rigid endoscope 71 has a structure in which an observation window 73 and irradiation windows 74-1 to 74-4 are arranged in the front end surface of a lens barrel 72.

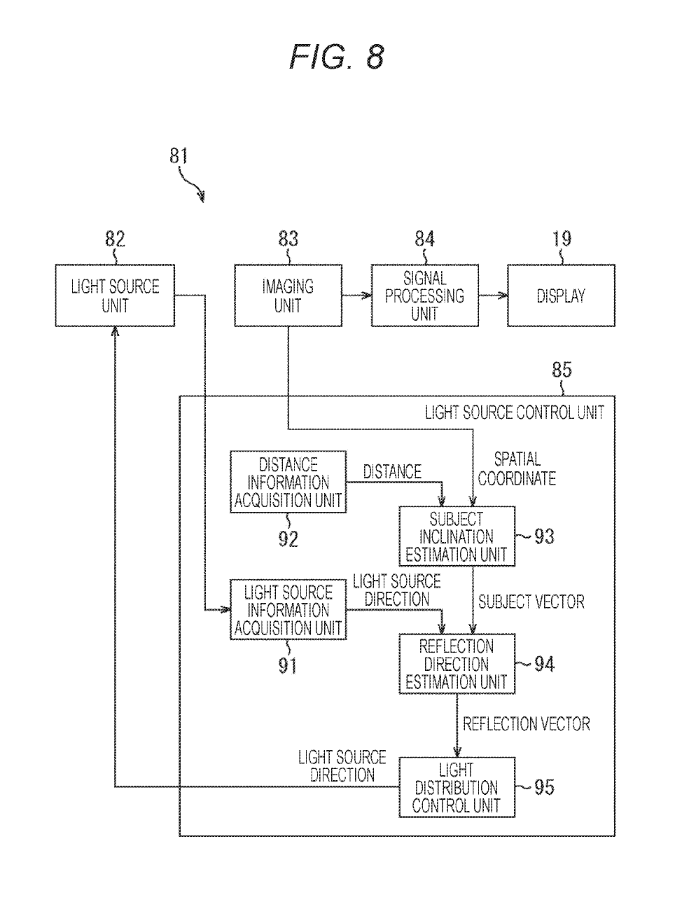

The observation window 73 is arranged at the center of the front end surface of the lens barrel 72. The light which is reflected by the subject and enters the observation window 73 is transmitted to the imaging unit (for example, imaging unit 83 in FIG. 8 to be described later), and an image is imaged by using the light.

The irradiation windows 74-1 to 74-4 are arranged at intervals of substantially 90° as viewing the lens barrel 72 from the front side, and the irradiation light can be emitted through each of the irradiation windows 74-1 to 74-4. Then, as described later, by adjusting the intensity of the irradiation light applied to the subject through the irradiation windows 74-1 to 74-4, the light source direction of the irradiation light is controlled.

The rigid endoscope 71 is formed in this way, and in the endoscopic surgical system 11, the light source of the irradiation light applied to the subject through the irradiation windows 74-1 to 74-4 is controlled.

FIG. 8 is a block diagram of an exemplary configuration of a light source control system 81 which controls the light source in the endoscopic surgical system 11.

As illustrated in FIG. 8, the light source control system 81 includes a light source unit 82, the imaging unit 83, a signal processing unit 84, and a light source control unit 85. For example, the light source control system 81 includes the camera head portion 12, the CCU 15, and the light source device 16 in FIG. 1, and an image is output from the light source control system 81 to the display 19.

The light source unit 82 has a plurality of light sources which generates the irradiation light irradiated from each of the irradiation windows 74-1 to 74-4 in FIG. 7. Then, the light source unit 82 adjusts the intensities of the light sources (for example, strong/weak and on/off) under the control by the light source control unit 85 to change the direction of the illumination light irradiating the subject.

The imaging unit 83 images an image by using the light entered from the observation window 73 in FIG. 7 and supplies an image signal obtained by the imaging to the signal processing unit 84.

The signal processing unit 84 performs signal processing to the image signal supplied from the imaging unit 83, and causes the display 19 to display the image to which the signal processing has been performed.

The light source control unit 85 includes a light source information acquisition unit 91, a distance information acquisition unit 92, a subject inclination estimation unit 93, a reflection direction estimation unit 94, and a light distribution control unit 95, and controls the light source unit 82.

The light source information acquisition unit 91 obtains light source information output from the light source unit 82 and supplies the current light source direction obtained from the light source information to the reflection direction estimation unit 94.

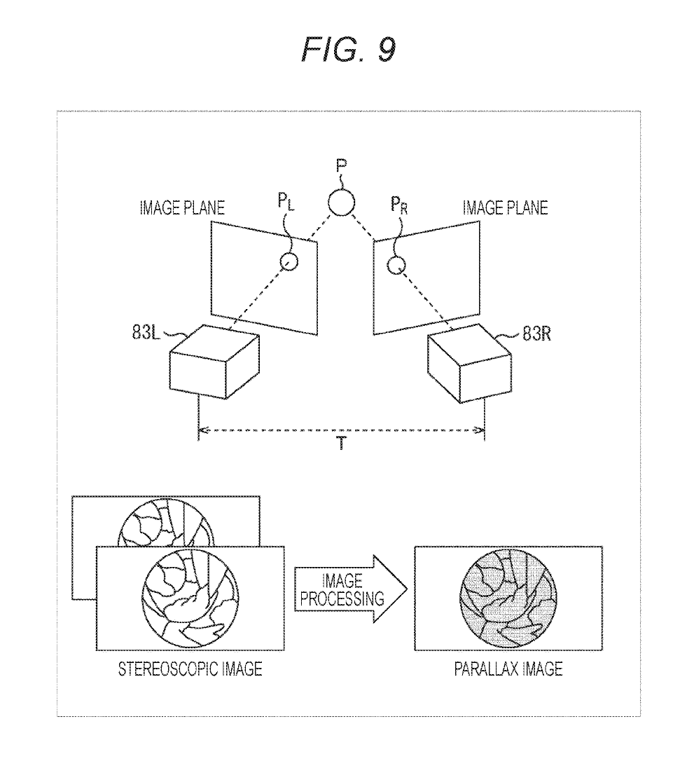

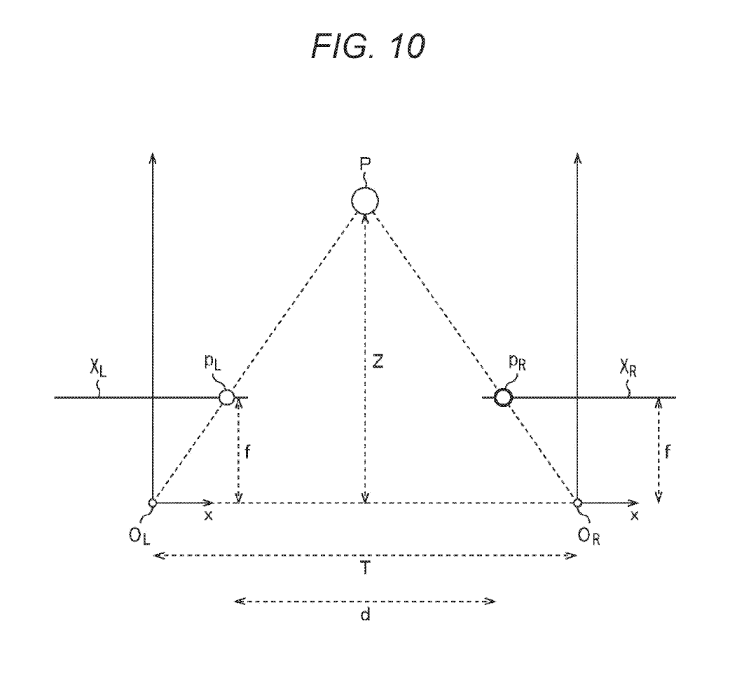

The distance information acquisition unit 92 obtains the distance information from the imaging unit 83 to the subject and supplies the distance indicated by the distance information to the subject inclination estimation unit 93. For example, as described with reference to FIGS. 9 and 10 to be described later, the distance information acquisition unit 92 can obtain the distance information with a method using stereoscopic image data and stereo matching. Furthermore, various methods can be used to obtain the distance information (for example, method of irradiating infrared rays in a time division or method using millimeter-wave radar). It is preferable that the distance information acquisition unit 92 obtain the distance information by using any one of the methods, and the obtaining method is not limited to the specific method.

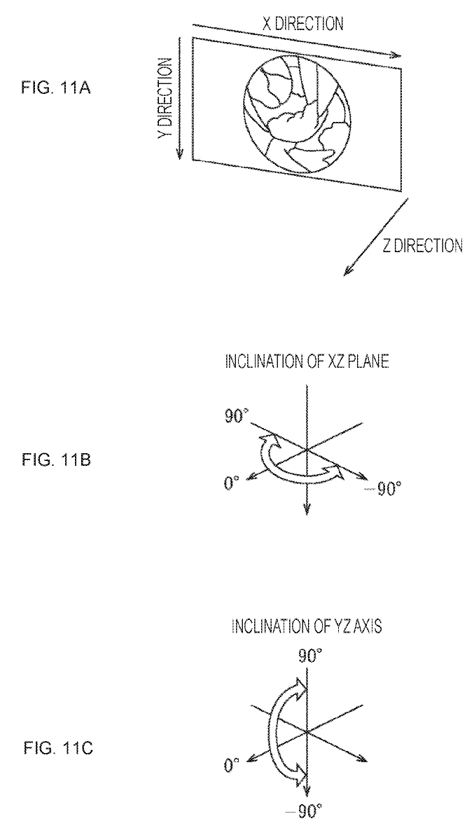

A spatial coordinate (two-dimensional coordinate) of the image imaged by the imaging unit 83 is supplied from the imaging unit 83 to the subject inclination estimation unit 93. Then, the subject inclination estimation unit 93 performs processing of estimating a three-dimensional inclination of the subject by using the spatial coordinate and the distance from the light source to the subject supplied from the distance information acquisition unit 92, and supplies a subject vector obtained as a result of the processing to the reflection direction estimation unit 94. Furthermore, a method of estimating the inclination of the subject by the subject inclination estimation unit 93 will be described with reference to FIG. 11 below.

The reflection direction estimation unit 94 performs processing of obtaining the reflection direction of the subject for each pixel or region by using the subject vector supplied from the subject inclination estimation unit 93 and the current direction of the light source supplied from the light source information acquisition unit 91, and supplies a reflection vector obtained as a result of the processing to the light distribution control unit 95.

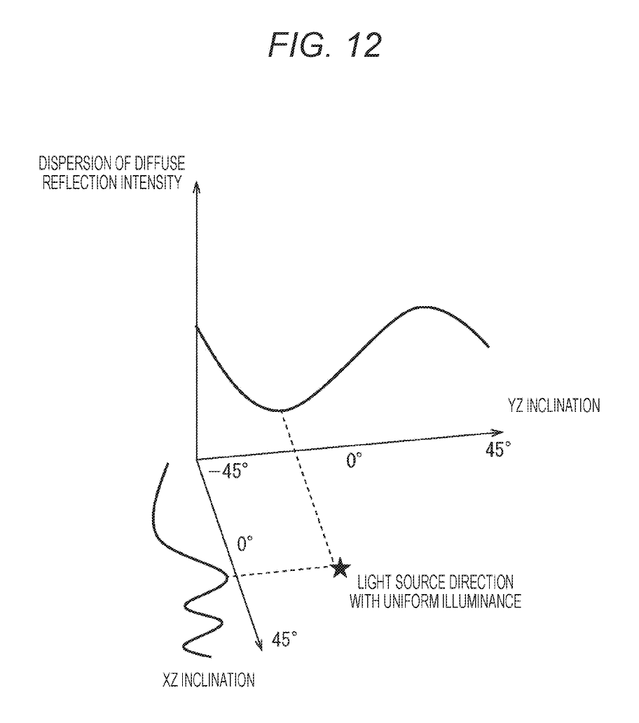

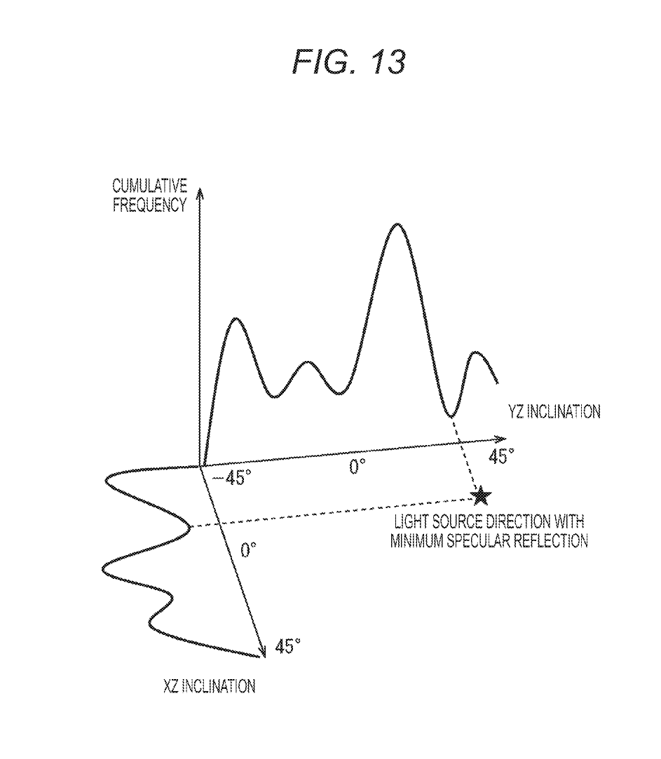

On the basis of the reflection vector supplied from the reflection direction estimation unit 94, the light distribution control unit 95 estimates a diffuse reflection intensity and obtains the light source direction with which the subject illuminance becomes uniform as viewed from the direction in which the subject is observed (that is, optical axial direction of imaging unit 83) (refer to FIG. 12) or the light source direction with which the specular reflection is minimized (refer to FIG. 13). Then, the light distribution control unit 95 controls the light distribution so that the subject is irradiated with the irradiation light from the irradiation windows 74-1 to 74-4 in FIG. 7 in the optimal light source direction obtained as described above.

The light source control system 81 is formed in this way, and can irradiate the subject with the irradiation light in the optimal light source direction on the basis of the reflection direction of the irradiation light on the surface of the subject and the direction in which the subject is observed. With this structure, the imaging unit 83 can image an image in a more appropriate exposure state.

With reference to FIGS. 9 and 10, as an exemplary method of obtaining the distance information from the imaging unit 83 to the subject, a method using the stereoscopic image data and the stereo matching will be described.

For example, as illustrated in FIG. 9, when a subject in a living body is imaged by using the rigid endoscope 71 in FIG. 7, by using two imaging units 83L and 83R, stereoscopic images imaged by the imaging units 83L and 83R are obtained. Then, by performing image processing to the stereoscopic images, a parallax image corresponding to an imaging interval T between the imaging units 83L and 83R is obtained.

Then, as illustrated in FIG. 10, a parallax d at the time when a point P in the real world is imaged by the imaging units 83L and 83R is expressed by the following formula (4) by using a horizontal coordinate x_{L} of a point p_{L} on an image plane X_{L} of the imaging unit 83L corresponding to the point P and a horizontal coordinate x_{R} of a point p_{R} on an image plane X_{R} of the imaging unit 83R corresponding to the point P.

[Formula 4]

d=x_{L}−x_{R} (4)

Furthermore, a relationship between a distance (depth) Z from the imaging units 83L and 83R to the point P in the real world and the imaging interval T between an imaging center O_{L} of the imaging unit 83L and an imaging center O_{R} of the imaging unit 83R is expressed by the following formula (5) by using the parallax d and a focal distance f.

$$\begin{matrix}[ Formula 5 ] & \\ \frac{T - d}{Z - f} = \frac{T}{Z} & ( 5 )\end{matrix}$$

Therefore, according to Formulas (4) and (5), the distance Z from the imaging units 83L and 83R to the point P in the real world is expressed by the following formula (6).

$$\begin{matrix}[ Formula 6 ] & \\ Z = \frac{f \times T}{{x}_{L} - {x}_{R}} & ( 6 )\end{matrix}$$

On the basis of such a stereoscopic image, the distance information acquisition unit 92 can obtain the distance from the imaging unit 83 to the subject. Then, the subject inclination estimation unit 93 can estimate the inclination of the subject with respect to the optical axial direction (depth direction) of the imaging unit 83 by using the distance from the imaging unit 83 to the subject.

With reference to FIG. 11, processing of estimating the inclination of the subject for each pixel or region from the distance information by the subject inclination estimation unit 93 will be described.

For example, as illustrated in A of FIG. 11, three-dimensional coordinates and axes are defined, and as illustrated in B of FIG. 11, an inclination of a XZ plane is defined. As illustrated in C of FIG. 11, an inclination of a YZ plane is defined. At this time, an angle θ₁ of the XZ plane and an angle θ₂ of the YZ plane can be obtained by the following formula (7) by using two predetermined target coordinates (x₁, y₁, z₁) and (x₂, y₂, z₂) on the imaging plane. Note that the two points used as the target coordinates to obtain the inclination may be two points specified by pixels or may be two regions specified by regions including the plurality of pixels.

$$\begin{matrix}[ Formula 7 ] & \\ \{ \begin{matrix}{θ}_{1} = {tan}^{- 1} ( \frac{{x}_{2} - {x}_{1}}{{z}_{2} - {z}_{1}} ) \\ {θ}_{2} = {tan}^{- 1} ( \frac{{y}_{2} - {y}_{1}}{{z}_{2} - {z}_{1}} )\end{matrix} & ( 7 )\end{matrix}$$

In this way, the subject inclination estimation unit 93 can obtain the inclination of the subject, and the reflection direction estimation unit 94 can estimate the reflection direction from the inclination of the subject.

That is, the reflection direction estimation unit 94 estimates the specular reflection intensity S and the diffuse reflection intensity I_{d} for each pixel or region of which the inclination is obtained by the subject inclination estimation unit 93.

For example, the reflection direction estimation unit 94 can estimate the diffuse reflection intensity I_{d} on the basis of a reflection component estimation mathematical model such as the Phong reflection model indicated in Formula (1), the inclination of the subject (angle θ₁ of XZ plane and angle θ₂ of YZ plane) obtained by Formula (7), and the light source direction supplied from the light source information acquisition unit 91.

Furthermore, similarly to the diffuse reflection intensity I_{d}, the reflection direction estimation unit 94 can estimate the intensity S of the specular reflection light on the basis of the mathematical model indicated in Formula (2), the inclination of the subject (angle θ₁ of XZ plane and angle θ₂ of YZ plane) obtained by Formula (7), and the light source direction supplied from the light source information acquisition unit 91.

At this time, the reflection direction estimation unit 94 can obtain, for example, the intensity Ii of incident light from the light source unit 82. Furthermore, for the diffuse reflectance K_{d}, the specular reflectance W, and the highlight coefficient n used in Formulas (1) and (2), arbitrary constants can be used, or a reflection coefficient unique for each subject may be used by identifying the subject by using image recognition and the like. In addition, the reflection model used at this time is not limited to the Phong reflection model described above as long as the reflection intensity can be estimated, and other various models can be used.

Next, FIGS. 12 and 13 are diagrams to describe a method of estimating an appropriate light source direction by the light distribution control unit 95.

With reference to FIG. 12, a method of estimating the light source direction so that the subject is uniformly illuminated will be described.

For example, as illustrated in FIG. 12, a reflection angle on the XZ plane is indicated by the X axis, a reflection angle on the YZ plane is indicated by the Y axis, and a dispersion value of the diffuse reflection intensity I_{d} is indicated by the Z axis. Then, the light distribution control unit 95 can estimate the light source direction with which the subject is uniformly illuminated by calculating the inclinations of the XZ plane and the YZ plane which can minimize the dispersion value of the diffuse reflection intensity I_{d}.

In this way, the light distribution control unit 95 estimates the light source direction so that the subject illuminance viewed from the imaging unit 83 becomes uniform so that the appropriate subject illumination can be realized.

With reference to FIG. 13, a method of estimating the light source direction so as to minimize the specular reflection will be described.

For example, the light distribution control unit 95 obtains a cumulative frequency for each angle of reflection from the reflection vector obtained by the reflection direction estimation unit 94 and obtains a light source vector which makes the frequency of generation of the specular reflection be the lowest. For example, a three-dimensional plot is illustrated in FIG. 13, an angle of reflection on the XZ plane is indicated by the X axis, an angle of reflection on the YZ plane is indicated by the Y axis, and a cumulative frequency of the angles is indicated by the Z axis. Then, the light distribution control unit 95 can estimate the light source direction which minimizes the specular reflection by calculating the inclinations of the XZ plane and the YZ plane to minimize the cumulative frequency in the movable range of the light source.

In this way, the light distribution control unit 95 estimates the light source direction which suppresses the specular reflection to the imaging unit 83 and minimizes the specular reflection. Accordingly, the appropriate subject illumination can be realized.

As described with reference to FIGS. 12 and 13, the light distribution control unit 95 can realize the appropriate subject illumination according to the uniformity of the subject illuminance and can realize the appropriate subject illumination according to the less specular reflectance. Note that, the light distribution control unit 95 may realize appropriate subject illumination by a method other than the above.

Note that when the light distribution control unit 95 determines the light source direction, there is a possibility that evaluations on both the uniform subject illuminance and the frequency of the specular reflection are not compatible. In this case, the light distribution control unit 95 can determine the light source direction which has the highest comprehensive evaluation regarding the above two points as an appropriate direction or determine the light source direction by weighting one of the above evaluations.

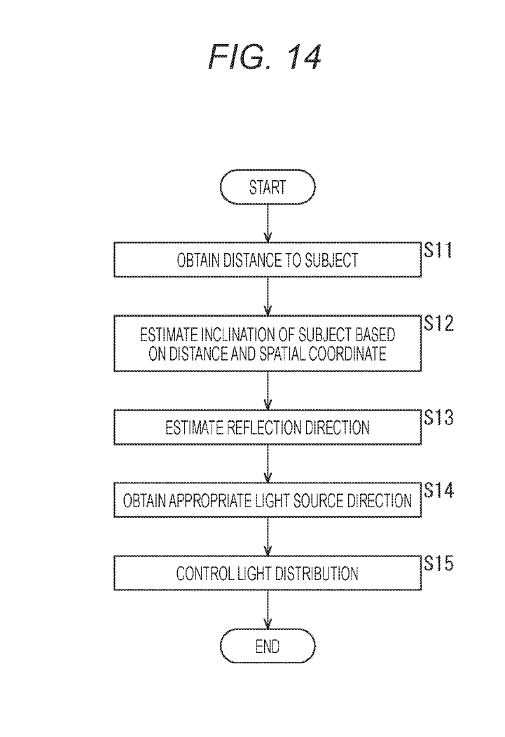

Next, FIG. 14 is a flowchart to describe light source control processing by the light source control unit 85 in FIG. 8.

For example, when an image is imaged by the imaging unit 83, the processing is started. In step S11, for example, as described above with reference to FIGS. 9 and 10, the distance information acquisition unit 92 obtains the distance from the imaging unit 83 to the subject and supplies the distance to the subject inclination estimation unit 93.

In step S12, on the basis of the distance supplied from the distance information acquisition unit 92 in step S11 and the spatial coordinate of the image imaged by the imaging unit 83, as described above with reference to FIG. 11, the subject inclination estimation unit 93 estimates the inclination of the subject. Then, the subject inclination estimation unit 93 supplies the subject vector indicating the inclination of the subject to the reflection direction estimation unit 94.

In step S13, the reflection direction estimation unit 94 estimates the reflection direction of the illumination light on the subject on the basis of the subject vector supplied from the subject inclination estimation unit 93 in step S12. Then, the reflection direction estimation unit 94 supplies the reflection vector indicating the reflection direction of the illumination light on the subject to the light distribution control unit 95.

In step S14, on the basis of the reflection vector supplied from the reflection direction estimation unit 94 in step S13, the light distribution control unit 95 obtains the optimal light source direction so as to uniform the subject illuminance or minimize the specular reflection.

In step S15, the light distribution control unit 95 supplies the optimal light source direction obtained in step S14 to the light source unit 82, and controls the light distribution so that the subject is irradiated with the irradiation light from the irradiation windows 74-1 to 74-4 (FIG. 7) in the optimal light source direction.

After the processing in step S15, the light source control processing is terminated. Then, the imaging unit 83 images an image of the subject irradiated with the illumination light with the light distribution controlled as described above, and the processing is in a standby state until a next image is supplied from the imaging unit 83.

As described above, since the light source control unit 85 can obtain the optimal light source direction by estimating the reflection components on the surface of the subject, it is possible to observe the subject with a more appropriate exposure state. That is, the light source control unit 85 can improve visibility at the time of observation by using the image imaged by the imaging unit 83 by eliminating the nonuniformity of the subject illuminance or decreasing the frequency of the specular reflection.

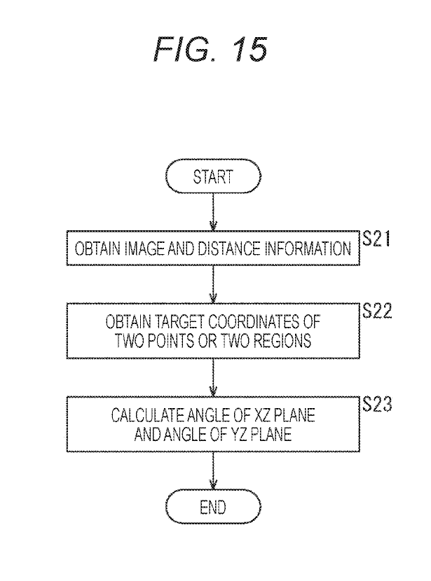

Next, FIG. 15 is a flowchart to describe processing of estimating the inclination of the subject by the subject inclination estimation unit 93.

In step S21, the subject inclination estimation unit 93 obtains the spatial coordinate of the image imaged by the imaging unit 83 and the distance information supplied from the distance information acquisition unit 92.

In step S22, the subject inclination estimation unit 93 obtains the target coordinates (x₁, y₁, z₁) and (x₂, y₂, z₂) which specify predetermined two points or two regions on the imaging plane.

In step S23, the subject inclination estimation unit 93 calculates the angle θ₁ of the XZ plane and the angle θ₂ of the YZ plane by using Formula (7) as described above with reference to FIG. 11. Then, the subject inclination estimation unit 93 supplies the inclination of the subject (angle θ₁ of XZ plane and angle θ₂ of YZ plane) to the reflection direction estimation unit 94, and the processing is terminated.

As described above, the subject inclination estimation unit 93 can estimate the inclination of the subject on the basis of the image and the distance information.

Here, in the processing of estimating the appropriate light source direction by the light distribution control unit 95, it is desirable to have three conditions, i.e., the direction has the small dispersion of the diffuse reflectance, has the low specular reflectance, and is close to the current light source direction. For example, when the endoscope image during a surgical operation or the like is observed as a moving image, a state should be avoided in which the impression of the subject (operated portion) is drastically and frequently changed according to the change in the illumination direction, and it is desirable that the light source direction be moderately changed with respect to the time and the space direction. Therefore, the above three conditions are required to observe the moving image.

On the other hand, in a case where a still image is imaged, since an optimal illumination condition at the imaging timing is desired, it is not necessary to satisfy the three conditions described above. For example, in this case, with conditions such that the dispersion of the diffuse reflectance is small and the specular reflectance is low, a new light source direction may be determined.

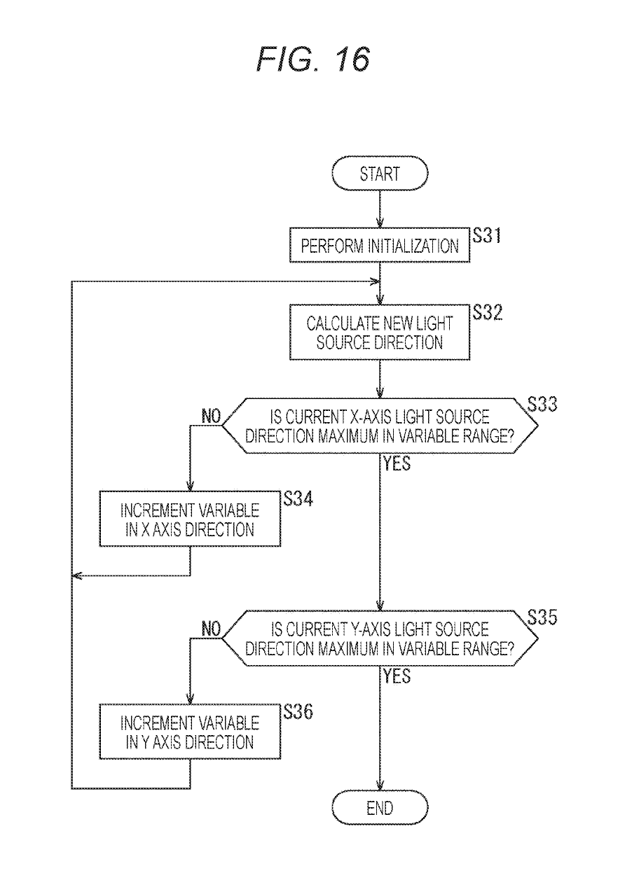

Furthermore, when the light distribution control unit 95 estimates an appropriate light source direction, the three conditions described above are independent factors. Therefore, the direction having the highest evaluation in each condition can be determined as a new light source direction. For example, as an example, description will be made with reference to a flowchart in FIG. 16.

FIG. 16 is a flowchart to describe processing of estimating the appropriate light source direction by the light distribution control unit 95.

In step S31, the light distribution control unit 95 performs initialization, sets a variable i in the X axis direction so that the X-axis light source direction is the minimum in a variable range n (i=X−n), and sets a variable j in the Y axis direction so that the Y-axis light source direction is the minimum in a variable range n (j=Y−n).

In step S32, the light distribution control unit 95 calculates a new light source direction D according to the following formula (8) by using a dispersion value Diff of the diffuse reflection component, a specular reflectance Spec, and a weight W of the distance. Furthermore, in Formula (8), the dispersion value Diff of the diffuse reflection component, the specular reflectance Spec, and the weight W of the distance have values from zero to one.

[Formula 8]

D=Max((1−Diff[i,j])×(1−Spec[i,j])×W[i,j], D) (8)

In step S33, the light distribution control unit 95 determines whether the current X-axis light source direction is the maximum (X+n) in the variable range n.

In step S33, in a case where the light distribution control unit 95 has determined that the current X-axis light source direction is not the maximum in the variable range n, the procedure proceeds to step S34, and the light distribution control unit 95 increments the variable i in the X axis direction (i=i+1). The procedure returns to step S32, and similar processing is repeated after that.

On the other hand, in step S33, in a case where the light distribution control unit 95 has determined that the current X-axis light source direction is the maximum in the variable range n, the procedure proceeds to step S35. That is, in this case, with the current Y-axis light source direction, the illumination light in all the X-axis light source directions from the minimum to the maximum in the variable range is irradiated.

In step S35, the light distribution control unit 95 determines whether the current Y-axis light source direction is the maximum (Y+n) in the variable range n.

In step S35, in a case where the light distribution control unit 95 has determined that the current Y-axis light source direction is not the maximum in the variable range n, the procedure proceeds to step S36, and the light distribution control unit 95 increments the variable j in the Y axis direction (j=j+1). After that the procedure returns to step S32, and similar processing is repeated.

On the other hand, in step S35, in a case where the light distribution control unit 95 has determined that the current Y-axis light source direction is the maximum in the variable range n, the processing is terminated. With this processing, the illumination light in all the X-axis light source directions and Y-axis light source directions in the variable range from the minimum to the maximum is irradiated, and the light source direction obtained in the final step S32 is the optimal light source direction.

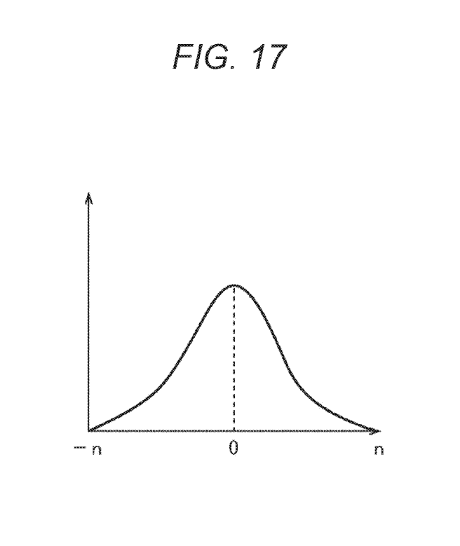

Furthermore, in such processing, the distance weight W can be determined on the basis of a relative angle with the new light source direction (Min=0, Max=n) with the current light source direction as a reference. For example, the preferable design is made by using the normal distribution (refer to FIG. 17) indicated by the following formula (9) so that the smaller the relative angle is, the distance weight is close to one and the larger the relative angle is, the distance weight is close to zero.

$$\begin{matrix}[ Formula 9 ] & \\ f ( w ) = ( \frac{1}{\sqrt{2 π} σ} {e}^{\frac{- {w}^{2}}{2 {σ}^{2}}} ) & ( 9 )\end{matrix}$$

Here, in Formula (9), as a weighting coefficient σ, a constant may be used, and a variable which is adaptable for the distance from the imaging unit 83 to the subject may be used. Furthermore, in a case where the weighting coefficient σ is used as a variable, it is preferable to appropriately determine the weighting coefficient σ from the distance information. For example, if the distance from the imaging unit 83 to the subject is long, the weighting coefficient σ is set to be small by largely moving an illuminated range by a slight change in the light source direction, and if the distance from the imaging unit 83 to the subject is short, the weighting coefficient σ is set to be large. In this way, more excellent observation can be performed.

As described above, in the endoscopic surgical system 11, by controlling the light source direction to decrease the dispersion of the diffuse reflectance or decrease the specular reflectance, for example, generation of an extremely bright portion in the image is avoided, and the observation can be performed with a more appropriate exposure state.

Note that, in the embodiment, the method of controlling the light source direction as the optical axis of the imaging unit 83 is fixed has been described. However, it is preferable to relatively adjust the optical axis of the imaging unit 83 and the light source direction, for example, the optical axis of the imaging unit 83 may be controlled while the light source direction is fixed. Furthermore, the method of controlling the light source direction is not limited to the method of controlling the irradiation intensity of the irradiation light emitted from each of the irradiation windows 74-1 to 74-4 in FIG. 7. Various methods (for example, method using movable light source, method of selecting appropriate light source from among a large number of light sources and the like) can be employed.

Furthermore, in addition to the endoscopic surgical system 11, for example, the present technology can be applied to various devices used to perform observation under an illumination environment similar to that of the endoscope.

Note that it is not necessary for the processing described above with reference to the flowcharts to be necessarily executed in time series in an order described in the flowchart, and the processing described above includes processing which is executed in parallel or processing which is individually executed (for example, parallel processing or processing by object). Furthermore, the program may be executed by a single CPU, and may be distributively processed by a plurality of CPUs. Furthermore, herein, the system indicates a whole device including a plurality of devices.

Furthermore, the above-mentioned series of processing (information processing method) can be performed by hardware and software. In a case where the software executes the series of processing, a program included in the software is installed from a program recording medium, in which the program has been recorded, to a computer installed in a dedicated hardware or, for example, a general-purpose personal computer which can perform various functions by installing various programs.

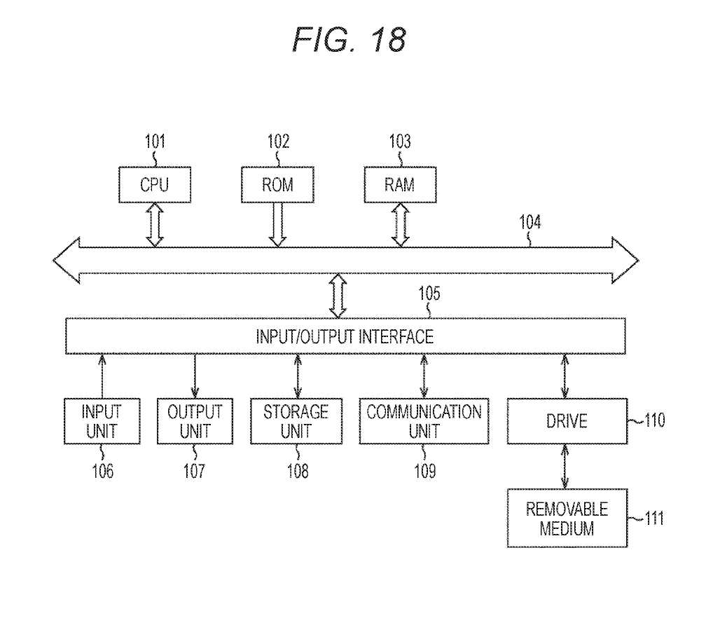

FIG. 18 is a block diagram of an exemplary configuration of hardware of the computer for executing the above-mentioned series of processing by the program.

In a computer, a central processing unit (CPU) 101, a read only memory (ROM) 102, and a random access memory (RAM) 103 are connected to each other with a bus 104.

In addition, an input/output interface 105 is connected to the bus 104. The input/output interface 105 is connected to an input unit 106 including a keyboard, a mouse, a microphone, and the like, an output unit 107 including a display, a speaker, and the like, a storage unit 108 including a hard disk, a nonvolatile memory, and the like, a communication unit 109 including a network interface, a drive 110 which drives a removable medium 111 such as a magnetic disk, an optical disk, a magneto-optical disk, or a semiconductor memory.

In the computer configured as above, the CPU 101 loads, for example, the program stored in the storage unit 108 to the RAM 103 via the input/output interface 105 and the bus 104 and executes the program so that the above-mentioned series of processing is performed.

The program executed by the computer (CPU 101) is provided, for example, by recording the program to the removable medium 111 which is a package medium such as a magnetic disk (including flexible disk), an optical disk (compact disc-read only memory (CD-ROM), a digital versatile disc (DVD), and the like), a magneto-optical disk, or a semiconductor memory or via a wired or wireless transmission medium such as a local area network, the Internet, or a digital satellite broadcast.

Then, the program can be installed to the storage unit 108 via the input/output interface 105 by mounting the removable medium 111 in the drive 110. Furthermore, the program can be received by the communication unit 109 via the wired or wireless transmission medium and installed to the storage unit 108. In addition, the program can be previously installed to the ROM 102 and the storage unit 108.

Note that, the present technology can have the configuration below.

(1) A light source control device including:

a reflection direction estimation unit configured to estimate a reflection direction in which irradiation light is reflected on a surface of a subject in a living body; and

a control unit configured to relatively control a direction of a light source for emitting the irradiation light relative to a direction in which the subject is observed on the basis of the reflection direction of the irradiation light estimated by the reflection direction estimation unit and the direction in which the subject is observed.

(2) The light source control device according to (1), in which

on the basis of the reflection direction, the control unit controls the direction of the light source so that an illuminance of the subject viewed from the direction in which the subject is observed becomes uniform.

(3) The light source control device according to (1), in which

on the basis of the reflection direction, the control unit controls the direction of the light source so that specular reflection in the direction in which the subject is observed is suppressed.

(4) The light source control device according to any one of (1) to (3), further including:

a subject inclination estimation unit configured to estimate an inclination of the subject relative to the direction in which the subject is observed, in which

the reflection direction estimation unit estimates the reflection direction on the basis of the inclination of the subject estimated by the subject inclination estimation unit.

(5) The light source control device according to (4), further including:

a distance information acquisition unit configured to obtain a distance from an imaging unit for imaging the subject to the subject, in which

the subject inclination estimation unit estimates the inclination of the subject on the basis of a spatial coordinate of an image imaged by the imaging unit and the distance obtained by the distance information acquisition unit.

(6) The light source control device according to (5), in which

the reflection direction estimation unit estimates the inclination of the subject for each pixel or region of the image imaged by the imaging unit.

(7) The light source control device according to any one of (1) to (6), in which

the subject in the living body is imaged by an endoscope.

(8) The light source control device according to (5), in which

the distance information acquisition unit obtains the distance by using stereoscopic images of the subject in the living body imaged by an endoscope.

(9) A light source control method including:

estimating a reflection direction in which irradiation light is reflected on a surface of a subject in a living body; and

relatively controlling a direction of a light source for emitting the irradiation light relative to a direction in which the subject is observed on the basis of the estimated reflection direction of the irradiation light and the direction in which the subject is observed.

(10) A program for causing a computer to execute steps including:

estimating a reflection direction in which irradiation light is reflected on a surface of a subject in a living body; and

relatively controlling a direction of a light source for emitting the irradiation light relative to a direction in which the subject is observed on the basis of the estimated reflection direction of the irradiation light and the direction in which the subject is observed.

(11) A surgical system including:

a light source configured to irradiate an operated portion in a living body with irradiation light from a predetermined direction;

an imaging unit configured to image the operated portion in the living body;

a reflection direction estimation unit configured to estimate a reflection direction in which the irradiation light is reflected on a surface of the operated portion; and

a control unit configured to relatively control a direction of the light source for emitting the irradiation light relative to an optical axial direction of the imaging unit on the basis of the reflection direction of the irradiation light estimated by the reflection direction estimation unit and the optical axial direction of the imaging unit.

In addition, the present embodiment is not limited to the embodiment described above and can be variously changed without departing from the scope of the present disclosure.

REFERENCE SIGNS LIST
11 endoscopic surgical system
12 camera head portion
13 energy treatment tool
14 forceps
15 CCU
16 light source device
17 treatment tool device
18 pneumoperitoneum device
19 display
20 recorder
21 printer
22a and 22b trocar
23 bed for patient
24 cart
25 foot switch
26 affected part
51 FPGA board
52 CPU
53-1 and 53-2 GPU board
54 memory
55 IO controller
56 recording medium
57 interface
58 bus
61 endoscope
62 lens barrel
63 illumination window
64 observation window
71 rigid endoscope
72 lens barrel
73 observation window
74-1 to 74-4 irradiation window
81 light source control system
82 light source unit
83 imaging unit
84 signal processing unit
85 light source control unit
91 light source information acquisition unit
92 distance information acquisition unit
93 subject inclination estimation unit
94 reflection direction estimation unit
95 light distribution control unit

1. A light source control device comprising:
a reflection direction estimation unit configured to estimate a reflection direction in which irradiation light is reflected on a surface of a subject in a living body; and
a control unit configured to relatively control a direction of a light source for emitting the irradiation light relative to a direction in which the subject is observed on the basis of the reflection direction of the irradiation light estimated by the reflection direction estimation unit and the direction in which the subject is observed.

2. The light source control device according to claim 1, wherein
on the basis of the reflection direction, the control unit controls the direction of the light source so that an illuminance of the subject viewed from the direction in which the subject is observed becomes uniform.

3. The light source control device according to claim 1, wherein
on the basis of the reflection direction, the control unit controls the direction of the light source so that specular reflection in the direction in which the subject is observed is suppressed.

4. The light source control device according to claim 1, further comprising:
a subject inclination estimation unit configured to estimate an inclination of the subject relative to the direction in which the subject is observed, wherein
the reflection direction estimation unit estimates the reflection direction on the basis of the inclination of the subject estimated by the subject inclination estimation unit.

5. The light source control device according to claim 4, further comprising:
a distance information acquisition unit configured to obtain a distance from an imaging unit for imaging the subject to the subject, wherein
the subject inclination estimation unit estimates the inclination of the subject on the basis of a spatial coordinate of an image imaged by the imaging unit and the distance obtained by the distance information acquisition unit.

6. The light source control device according to claim 5, wherein
the reflection direction estimation unit estimates the inclination of the subject for each pixel or region of the image imaged by the imaging unit.

7. The light source control device according to claim 1, wherein
the subject in the living body is imaged by an endoscope.

8. The light source control device according to claim 5, wherein
the distance information acquisition unit obtains the distance by using stereoscopic images of the subject in the living body imaged by an endoscope.

9. A light source control method comprising:
estimating a reflection direction in which irradiation light is reflected on a surface of a subject in a living body; and
relatively controlling a direction of a light source for emitting the irradiation light relative to a direction in which the subject is observed on the basis of the estimated reflection direction of the irradiation light and the direction in which the subject is observed.

10. A program for causing a computer to execute steps comprising:
estimating a reflection direction in which irradiation light is reflected on a surface of a subject in a living body; and
relatively controlling a direction of a light source for emitting the irradiation light relative to a direction in which the subject is observed on the basis of the estimated reflection direction of the irradiation light and the direction in which the subject is observed.

11. A surgical system comprising:
a light source configured to irradiate an operated portion in a living body with irradiation light from a predetermined direction;
an imaging unit configured to image the operated portion in the living body;
a reflection direction estimation unit configured to estimate a reflection direction in which the irradiation light is reflected on a surface of the operated portion; and
a control unit configured to relatively control a direction of the light source for emitting the irradiation light relative to an optical axial direction of the imaging unit on the basis of the reflection direction of the irradiation light estimated by the reflection direction estimation unit and the optical axial direction of the imaging unit.
